(19) United States
(12) Patent Application Publication
(10) Pub. No.: US 2005/0095233 A1
(43) Pub. Date: May 5, 2005

(54) Composition and method for reducing lipid storage

(76) Inventors: Edward Larry McCleary, Incline Village, NV (US); Carl A. Forest, Boulder, CO (US); Christine McCleary, Incline Village, NV (US)

Correspondence Address: PATTON BOGGS, 1660 LINCOLN ST, SUITE 2050, DENVER, CO, 80264, US

(21) Appl. No.: 10/987,108

(22) Filed: Nov. 12, 2004

Related U.S. Application Data
(63) Continuation-in-part of application No. 10/462,958, filed on Jun. 17, 2003.
Application No. 10/462,958 is a continuation-in-part of application No. 09/749,584, filed on Dec. 28, 2000, now Pat. No. 6,579,866.
(63) Continuation-in-part of application No. 10/890,067, filed on Jul. 12, 2004.
Application No. 10/890,067 is a continuation-in-part of application No. 10/462,958, filed on Jun. 17, 2003.
Application No. 10/890,067 is a continuation-in-part of application No. 10/616,674, filed on Jul. 10, 2003.
(60) Provisional application No. 60/520,466, filed on Nov. 14, 2003.
(60) Provisional application No. 60/536,286, filed on Jan. 13, 2004.

(51) Int. Cl. A61K 38/43; A61K 31/555; A61K 31/4415; A61K 31/4188; A61K 31/198

(52) U.S. Cl. 424/94.1; 514/383; 514/554; 514/561; 514/574; 514/350; 514/184; 514/400; 514/423; 514/560; 424/762; 424/776; 514/263.31; 514/171; 514/419

(57) ABSTRACT

An orally or parenterally administered composition for reducing the storage of lipids in a human, the composition comprising: an effective amount of hydroxycitric acid; an effective amount of carnitine; an effective amount of biotin; an effective amount of one or more gluconeogenic substrates selected from the group consisting of: aspartate, lactate, glycerol, and a gluconeogenic amino acid or alphaketo analogue thereof; an effective amount of eicosapentanoic acid; and an effective amount of one or more ingredients selected from the group consisting of: medium chain triglycerides (MCT) with fatty acid backbones containing 6 to 14 carbon atoms or their individual fatty acid analogues or metabolic precursors, sesame seeds or derivative products, sesamin and/or its epimer episesamin, caffeine, forskolin, 7-keto dehydroepiandrosterone (7-keto DHEA), green tea extract containing epigallocatechingallate (EGCG), capsaicum, and 5-hydroxytryptophan (5-HTP). The supplement may also be combined with a food, beverage, condiment, spice or salad dressing base to provide a food, beverage, condiment, spice or salad dressing product designed to reduce lipid storage.

### RELATED APPLICATIONS

This application is a continuation-in-part application of U.S. patent application Ser. No. 10/462,958 filed Jun. 17, 2003 in the name of Edward Larry McCleary, which itself is a continuation-in-part of U.S. patent application Ser. No. 09/749,584, filed Dec. 28, 2000 in the name of Edward Larry McCleary, now U.S. Pat. No. 6,579,866, which issued Jun. 17, 2003. This application is also a continuation-in-part of U.S. patent application Ser. No. 10/890,067 filed Jul. 12, 2004, which itself is a continuation-in-part of U.S. patent application Ser. No. No. 10/462,958 filed on Jun. 17, 2003 and is also a continuation-in-part of U.S. patent application Ser. No. 10/616,674 filed Jul. 10, 2003. Through the above-mentioned application Ser. No. 10/890,067, this application also claims the benefit of U.S. Provisional Patent Application No. 60/520,466 filed Nov. 14, 2003 and U.S. Provisional Patent Application Ser. No. 60/536,286 filed Jan. 13, 2004. All of the above-cited patent applications and patent are incorporated in this disclosure to the same extent as though fully disclosed herein.

### BACKGROUND OF THE INVENTION

‘2. Field of the Invention

This invention relates in general to nutraceuticals, including dietary supplements, nutritional supplements, medical foods, botanical drugs and drugs. In particular, it relates to a neutraceutical that promotes the reduction of intra and/or intercellular lipid storage, more particularly, a neutraceutical that is highly effective for weight loss. In another aspect, the invention also relates in particular to the combination of such nutraceuticals with foods, beverages, spices, condiments and salad dressings to form a functional food. 2. Statement of the Problem

As discussed in the above-mentioned patent applications and patent, which are not prior art to this application, disorders of nutrient partitioning form the basis for a group of metabolic disorders associated with excess intracellular lipid storage. These include, but are not limited to, insulin resistance, hyperinsulinemia, Syndrome X, hypertriglyceridemia and/or low HDL syndrome, high RQ (respiratory quotient) syndrome, obesity, chronic fatigue syndrome, small dense LDL syndrome, recidivism from weight loss, glucolipoxia, premature aging, memory loss, endothelial dysfunction, vascular disease, hypertension, postprandial hyperlipidemia, certain types of cancer, metabolic inflexibility, and others. The basic abnormality is similar in each circumstance but manifests clinically in different ways depending upon the organ involved, the individual's genetic makeup, age, sex, and other factors.

The two major macronutrient fuels are fat and carbohydrate (which is stored in the body as glycogen). In the body, fat and carbohydrate are combined in certain proportions to generate the fuel mix the body burns at any point in time. If the fuel mix contains more carbohydrate, it contains relatively less fat and vice versa. Because there is minimal metabolic transformation between carbohydrate and fat, if more fat is being burned, then less is being stored and vice versa. The same holds true for carbohydrate, i.e., if more carbohydrate is being burned, then less is being stored and vice versa.

The relative ratio of fat and carbohydrate contributing to the fat/carbohydrate fuel mix is referred to as the “respiratory quotient” (RQ). RQ is approximately 1 when only carbohydrate is being burned and is about 0.8 when fat is the sole component being oxidized. A mixture of fuels is assigned a number between approximately 0.8 and 1.0. The nutrient partitioning abnormality most frequently encountered as manifested by an elevated RQ indicates excessive fat storage associated with excessive combustion of carbohydrate. This condition predisposes to increased intracellular lipid stores in numerous organ systems as well as the entire body. The most affected organs are the liver, pancreas, adipose tissue, and skeletal muscle. Also involved is the hypothalamic-pituitary-adrenal axis as well as the vascular wall. When lipid stores accumulate in these organs, they produce alterations in intracellular signaling systems including insulin and protein kinase C (PKC) signaling pathways leading to skeletal muscle insulin resistance, excessive basal insulin secretion by the pancreas associated with a decrement in glucose-induced insulin release, an impairment of insulin action at the level of the liver manifested by decreased sensitivity of insulin suppression of hepatic glucose output, expansion of (visceral) fat stores, excessive cortisol secretion, and endothelial dysfunction associated with altered nitric oxide physiology. The defects primarily involving insulin signaling pathways act as predisposing factors which increase tissue glycation and oxidative stress. This constellation of abnormalities combines in various ways to form the basis for the metabolic diseases mentioned above.

Approximately 30% to 50% of the population of westernized societies manifest overtly abnormal fuel homeostasis. As a result, affected individuals have a predisposition to accumulate intracellular lipid in various tissues and organs, which alters the intracellular metabolic milieu and induces alterations in various strategic signal transduction systems, as discussed above. Such alterations induce different metabolic and physiologic alterations among individuals. In one person, obesity might result, while another may develop hypertension, non-insulin dependent diabetes mellitis (NIDDM), dyslipidemia, or vascular disease depending upon specific genetic interactions. Other people might evolve symptoms based upon oxidative stress and enhanced tissue glycation, causing external signs of premature aging or memory loss. In some circumstances, the primary abnormality may be only augmented IGF effects, leading to the development of prostatic hypertrophy or frank prostate cancer in an individual.

It is desirable, therefore, to provide a neutraceutical composition and method for modulating (i.e., altering or normalizing) aberrant pathways of nutrient partitioning which play a key role in numerous metabolic disorders. More particularly, it is desirable to provide a composition and method for modulating aberrant pathways of nutrient partitioning so as to avoid excessive fat storage and/or excessive carbohydrate oxidation. Specifically, it is desirable to provide a means for modulating aberrant pathways of nutrient partitioning so as to promote oxidation of fat and other lipids. It would be particularly desirable if the composition and method had little or no side effects.

Thus, it would also be highly desirable to have such nutraceuticals available in a manner that made them transparently easy to use in the course of everyday life, and at a time and quantity that was more compatible with human metabolism.

### SUMMARY OF THE INVENTION

The above-cited U.S. Pat. No. 6,579,866 disclosed and claimed an orally or parenterally administered composition for modulating nutrient partitioning in a human so as to increase oxidation of fat and increase storage of glycogen, the composition comprising: an effective amount of the hydroxycitric acid; an effective amount of carnitine; an effective amount of biotin; and an effective amount of one or more gluconeogenic substrates selected from the group consisting of: aspartate, lactate, glycerol, and a gluconeogenic amino acid or alphaketo analogue thereof; and an effective amount of eicosapentanoic acid. It also disclosed and claimed a method for modulating nutrient partitioning in a human so as to increase oxidation of fat and increase storage of glycogen, comprising orally or parenterally administering the foregoing composition to the human for a therapeutically effective period.

A double-blind clinical trial of the above composition showed it to be more effective than the best pharmaceutical in effecting weight loss and related health improvements, without the side effects associated with the pharmaceutical. Further studies have indicated that the composition can be improved upon by including one or more ingredients that provide enhanced fatty acid oxidation, enhancement of thermogenic futile cycles, and suppression of neural appetite centers.

As explained more fully in the above-cited application and patent, the compositions according to the invention work by interacting with the enzymes and other chemicals that regulate specific natural body functions. A key bodily function that is regulated is the oxidation of fat, which occurs naturally in all cells, but especially in the liver. One category of additional ingredients upregulates the burning of fatty acids. However, when fatty acids are oxidized, high energy electrons are created. The high energy electrons can follow at least two natural pathways in the body cells. One pathway utilizes the high energy electrons to synthesize ATP, which is stored in the body. When a cell's ability to store ATP is saturated, the cell ceases oxidizing fat. Thus, this pathway ultimately limits the amount of fat that can be oxidized. Another pathway takes the high energy electrons along a futile cycle in which they drop down to lower energy levels, and release heat. This pathway places no inherent limit on the amount of fatty acid that can be oxidized. A second category of additional ingredients upregulates this futile cycle. A third category of additional ingredients downregulates the appetite centers.

The additional ingredients that upregulate the burning of fatty acids include: medium chain triglycerides (MCT) with fatty acid backbones containing 6 to 14 carbon atoms or their individual fatty acid analogues or metabolic precursors; sesame seeds or their derivatives, sesamin and its epimer, episesamin; caffeine, and forskolin. The additional ingredients that upregulate the free electron futile cycle include: 7-keto dehydroepiandrosterone (7-keto DHEA), green tea extract containing epigallocatechingallate (EGCG), and capsaicum. The additional ingredients that downregulate the appetite centers include 5-hydroxytryptophan (5-HTP). The invention contemplates that one or more of the above ingredients are added to the compositions disclosed in the above-cited patent application and patent, and particularly to the compositions and methods claimed in the patent.

The invention provides composition for reducing the storage of lipids in a human, the composition comprising: an effective amount of the hydroxycitric acid; an effective amount of carnitine; an effective amount of biotin; an effective amount of one or more gluconeogenic substrates selected from the group consisting of: aspartate, lactate, glycerol, and a gluconeogenic amino acid or alphaketo analogue thereof; an effective amount of eicosapentanoic acid; and an effective amount of one or more ingredients selected from the group consisting of: medium chain triglycerides (MCT) with fatty acid backbones containing 6 to 14 carbon atoms, their individual fatty acid analogues or metabolic precursors, sesame seeds or their derivatives, sesamin and/or its epimer episesamin, caffeine, forskolin, 7-keto dehydroepiandrosterone (7-keto DHEA), green tea extract containing epigallocatechingallate (EGCG), capsaicum, and 5-hydroxytryptophan (5-HTP). Preferably, the composition comprises: a weight ratio of the hydroxycitric acid to the carnitine of from about 1:10 to about 100:1; a weight ratio of the hydroxycitric acid to the gluconeogenic substrate of from about 5:1 to about 1:60; and a weight ratio of the hydroxycitric acid to the biotin of from about 50:1 to about 2500:1. Preferably, the gluconeogenic amino acid is selected from the group consisting of: alanine, arginine, asparagine, cystine, glutamine, glycine, histidine, hydroxyproline, methionine, proline, serine, threonine, and valine. Preferably, the composition further comprises an effective amount of at least one nutritional supplement. Preferably, the nutritional supplement is selected from the group consisting of: chromium, conjugated linoleic acid, coenzyme Q10, pyridoxine, alpha-lipoic acid, magnesium, and gymnema sylvestre. Preferably, the composition comprises from about 0.2 grams to about 8 grams of hydroxycitric acid, from about 10 milligrams to about 10 grams of carnitine, from about 1 gram to about 75 grams of the gluconeogenic substrate; from about 1 milligram to about 25 milligrams of biotin, from about 100 micrograms to about 2 milligrams of chromium, from about 5 milligrams to about 500 milligrams of coenzyme Q10, from about 50 milligrams to about 20 grams of conjugated linoleic acid, from about 10 milligrams to about 10 grams of eicosapentaenoic acid, from about 25 milligrams to about 400 milligrams of pyridoxine, from about 25 milligrams to about 2000 milligrams of alpha lipoic acid, from about 200 milligrams to about 1600 milligrams of magnesium, and from about 20 milligrams to about 2000 milligrams of gymnemic acid. Preferably, the composition comprises: from about 0.5 grams to about 5 grams of hydroxycitric acid, from about 50 milligrams to about 5 grams of carnitine, from about 1 gram to about 30 grams of the gluconeogenic substrate, from about 2 milligrams to about 10 milligrams of biotin, from about 400 micrograms to about 1000 micrograms of chromium, from about 20 milligrams to about 300 milligrams of coenzyme Q10, from about 1 gram to about 10 grams of conjugated linoleic acid, from about 50 milligrams to about 5000 milligrams of eicosapentaenoic acid, from about 100 milligrams to about 300 milligrams of pyridoxine, from about 50 milligrams to about 1200 milligrams of alpha lipoic acid, from about 400 milligrams to about 1200 milligrams of magnesium, and from about 75 milligrams to about 500 milligrams of gymnemic acid. Preferably, the composition further comprises a pharmaceutically acceptable carrier.

The invention also provides a method for reducing the storage of lipids in a human, comprising orally or parenterally administering to the human, for a therapeutically effective period, a composition comprising: an effective amount of the hydroxycitric acid; an effective amount of carnitine; an effective amount of biotin; and an effective amount of one or more gluconeogenic substrates selected from the group consisting of: aspartate, lactate, glycerol, and a gluconeogenic amino acid or alphaketo analogue thereof; an effective amount of eicosapentanoic acid; and an effective amount of one or more ingredients selected from the group consisting of: medium chain triglycerides (MCT) with fatty acid backbones containing 6 to 14 carbon atoms or their individual fatty acid analogues or metabolic precursors, sesame seeds or their derivatives, sesamin and/or its epimer episesamin, caffeine, forskolin, 7-keto dehydroepiandrosterone (7-keto DHEA), green tea extract containing epigallocatechingallate (EGCG), capsaicum, and 5-hydroxytryptophan (5-HTP). Preferably, the composition is administered on a daily basis to the human. Preferably, the therapeutically effective period of time is at least three weeks. Preferably, the effective amount of the composition is at least about 1 gram per serving. Preferably, the effective amount of the composition is from about 4 grams to about 20 grams per serving. Preferably, the composition comprises: a weight ratio of the hydroxycitric acid to the carnitine of from about 1:10 to about 100:1; a weight ratio of the hydroxycitric acid to the gluconeogenic substrate of from about 5:1 to about 1:60; and a weight ratio of the hydroxycitric acid to the biotin of from about 50:1 to about 2500:1. Preferably, the gluconeogenic amino acid is selected from the group consisting of: alanine, arginine, asparagine, cystine, glutamine, glycine, histidine, hydroxyproline, methionine, proline, serine, threonine, and valine. Preferably, the composition further comprises an effective amount of at least one nutritional supplement. Preferably, the nutritional supplement is selected from the group consisting of: chromium, conjugated linoleic acid, coenzyme Q10, pyridoxine, alpha lipoic acid, magnesium, and gymnema sylvestre. Preferably, the composition comprises: from about 0.2 grams to about 8 grams of hydroxycitric acid, from about 10 milligrams to about 10 grams of carnitine, from about 1 gram to about 75 grams of the gluconeogenic substrate, from about 1 milligram to about 25 milligrams of biotin, from about 100 micrograms to about 2 milligrams of chromium, from about 5 milligrams to about 500 milligrams of coenzyme Q10, from about 50 milligrams to about 20 grams of conjugated linoleic acid, from about 10 milligrams to about 10 grams of eicosapentaenoic acid, from about 25 milligrams to about 400 milligrams of pyridoxine, from about 25 milligrams to about 2000 milligrams of alpha lipoic acid, from about 200 milligrams to about 1600 milligrams of magnesium, and from about 20 milligrams to about 2000 milligrams of gymnemic acid. Preferably, the composition comprises: from about 0.5 grams to about 5 grams of hydroxycitric acid, from about 50 milligrams to about 5 grams of carnitine, from about 1 gram to about 30 grams of the gluconeogenic substrate; from about 2 milligrams to about 10 milligrams of biotin, from about 400 micrograms to about 1000 micrograms of chromium, from about 20 milligrams to about 300 milligrams of coenzyme Q10, from about 1 gram to about 10 grams of conjugated linoleic acid, from about 50 milligrams to about 5000 milligrams of eicosapentaenoic acid, from about 100 milligrams to about 300 milligrams of pyridoxine, from about 50 milligrams to about 1200 milligrams of alpha lipoic acid, from about 400 milligrams to about 1200 milligrams of magnesium, and from about 75 milligrams to about 500 milligrams of gymnemic acid. Preferably, the composition further comprises a pharmaceutically acceptable carrier. Preferably, the method further includes having the human follow a dietary regimen involving a glycemic index of less than 60 and daily calorie consumption comprising less than 50% of calories from carbohydrate intake and at least 20% of calories from protein intake. Preferably, the method further includes having the human follow an exercise program involving aerobic and resistance training. Preferably, the method further involves having the human donate blood so as to produce a fall in serum ferritin levels and iron stores. Preferably, the method further involves having the human follow a stress reduction program so a to diminish glycocorticoid activity.

The invention also provides a solution to the above problem by systematically incorporating such nutraceuticals into foods, condiments and spices, salad dressings and beverages, preferably into certain foods that have become an everyday part of the modern diet. In the more preferred embodiments, the nutraceuticals are incorporated into foods that are normally eaten by persons having the chronic problems that the nutraceuticals address. In the most preferred embodiment, details of the metabolic process are used to select combinations of a food, a condiment, a spice, a beverage, or a salad dressing with nutraceuticals that will most likely address particular chronic dietary, nutritional or health problems mentioned above as well as other chronic health issues.

The invention provides a weight loss food or a lipid reduction food comprising: a food base; the above-described nutraceutical composition, which nutraceutical composition is present in an amount in excess of the amount of the nutraceutical composition present naturally in foods. In other aspects, a beverage base, a condiment or spice base, or a salad dressing base can be used instead of the food base.

The invention provides a food, beverage, condiment, spice or salad dressing product designed to reduce lipid storage, the product comprising: a food, beverage, condiment, spice or salad dressing base; and a nutraceutical composition comprising: an effective amount of the hydroxycitric acid; an effective amount of carnitine; an effective amount of biotin; an effective amount of one or more gluconeogenic substrates selected from the group consisting of: aspartate, lactate, glycerol, and a gluconeogenic amino acid or alphaketo analogue thereof; an effective amount of eicosapentanoic acid; and an effective amount of one or more ingredients selected from the group consisting of: medium chain triglycerides (MCT) with fatty acid backbones containing 6 to 14 carbon atoms or their individual fatty acid analogues or metabolic precursors, sesame seeds or their derivative products, sesamin and/or its epimer episesamin, caffeine, forskolin, 7-keto dehydroepiandrosterone (7-keto DHEA), green tea extract containing epigallocatechingallate (EGCG), capsaicum, 5-hydroxytryptophan (5-HTP); the supplement present in an amount in excess of the amount of the nutraceutical composition naturally present in the food, beverage, condiment, spice or salad dressing base. In one aspect, the base is a beverage base and the beverage is preferably an alcoholic beverage. In another aspect, the base is a food base and the food is preferably selected from the group consisting of: vegetables and vegetable food products such as baked beans, tomato-based products like tomato paste, stewed tomatoes, spaghetti sauce, pizza sauce, vegetable oils, fruit and fruit food products such as jellies, jams and syrups, cereals, trail mix, cookies, pasta, flours including wheat, soy, oat, and potato flour, whey, chocolate, candy, tofu, bagels, baked goods such as bread, cakes, pizza dough and mixes for these, pancakes and waffles and mixes for these, soups, trail mix, nutritional bars, peanut butter, potato chips, corn chips, crackers, meats, and meat food products, such as lunch meats, hot dogs and sausage, milk food products, such as ice cream, yogurt, cheese and butter. In another aspect, the base is a beverage base and the beverage is preferably selected from the group consisting of: soft drinks, tea, coffee, milk, fruit juices, and sports drinks. In a further aspect, the base is a condiment or spice base and the condiment or spice is preferably selected from the group consisting of: steak sauce, mustard, catsup, soy sauce, chili sauce, chip dips, salsa, pickles, horseradish, curry, chili powder, salt, pepper, vinegar, cinnamon, garlic and garlic powder, garlic oil, onion and onion powder and onion oil. Preferably, the gluconeogenic amino acid is selected from the group consisting of: alanine, arginine, asparagine, cystine, glutamine, glycine, histidine, hydroxyproline, methionine, proline, serine, threonine, and valine. Preferably, the composition further comprises an effective amount of at least one nutritional supplement. Preferably, the nutritional supplement is selected from the group consisting of: chromium, conjugated linoleic acid, coenzyme Q10, pyridoxine, magnesium, and gymnema sylvestre.

### DETAILED DESCRIPTION OF THE INVENTION

1. Overview

The invention relates to certain compositions which are of benefit to human well being. In general, we shall label these compositions as “nutraceuticals” herein. Depending the point of view of the persons using them, nutraceuticals may be called, dietary supplements, nutritional supplements, botanical drugs, medical foods, and sometimes even drugs. In the future, other terms may be applied to these compounds. The invention is intended to cover these compositions when used for the purposes stated, no matter what they are called.

As stated above, the present invention provides a method and composition for modulating nutrient partitioning so as to reduce excess lipid storage. As used herein, the term “modulating” with respect to the effect of the composition and method of this invention on nutrient partitioning means that the composition and method have a beneficial effect on the ratio of fat and carbohydrate in the fuel mix discussed previously herein, and, in particular, increase the oxidation of fat. More specifically, the term “modulating” means that the composition and method of this invention alter or normalize nutrient partitioning so as to increase fat oxidation and/or glycogen storage, which in turn reduces the RQ value (i.e., more fat and less carbohydrate is burned).

The method of the invention involves the daily administration of an effective amount of the composition preferably in conjunction with a specific dietary plan in a synergistic format. The method may include an exercise program and/or stress reduction program and/or blood donation program.

The composition of this invention is composed of effective amounts of: HCA, carnitine, biotin, and a gluconeogenic substrate preferably selected from one or more of the following: aspartate, lactate, glycerol, and any gluconeogenic amino acid or its alpha-keto analogue, plus additional ingredients to be discussed below. The gluconeogenic amino acid is preferably alanine, arginine, asparagine, cystine, glutamine, glycine, histidine, hydroxyproline, methionine, proline, serine, threonine and valine.

Preferably, the composition of this invention further contains one or more of the following: chromium, CLA, coenzyme Q10, EPA (either alone or as part of fish oil), pyridoxine, alpha lipoic acid, magnesium, and gymnema sylvestre.

HCA and carnitine promote hepatic fatty acid oxidation by activating carnitine palmitoyl transferase (CPT), the rate-limiting enzyme in the fatty acid oxidation process.

EPA (either alone or as part of fish oil) inhibits the enzymes citrate lyase (CL) and acetyl CoA carboxylase (ACC), thus inhibiting the production of malonyl CoA—an allosteric inhibitor of CPT. EPA/fish oil thus acts to disinhibit fatty acid oxidation.

As one of its metabolic effects, CLA promotes fat-to-lean partitioning changes via the activation of CPT.

Thus, HCA, carnitine, EPA, and CLA promote or disinhibit hepatic fatty acid oxidation.

Hepatic fat oxidation produces reducing equivalents, ATP, and acetyl CoA which drive hepatic gluconeogenesis. The elevated acetyl CoA levels activate the liver enzyme pyruvate carboxylase (PC) and high ATP levels inhibit pyruvate dehydrogenase (PDH). These effects synergistically accelerate hepatic gluconeogenesis while simultaneously inhibiting fat synthesis. The reducing equivalents generated by disinhibited fatty acid oxidation are consumed in the reductive synthetic processes of gluconeogenesis.

Hepatic gluconeogenesis acts via the indirect route to expand liver glycogen stores and provides a slow continuous time release source of glucose from the liver. Both of these have the physiologic effect of profoundly suppressing appetite and increasing energy. These effects are enhanced by the consumption of appropriate amounts of low glycemic index carbohydrates whose glucose content is absorbed slowly over a prolonged time interval, thus acting as a sustained release glucose source emanating from the gut.

It has also been shown that hepatic triglyceride (TG) synthesis as well as very low density lipoprotein (VLDL) secretion are diminished coincidentally with increasing hepatic fatty acid (FA) oxidation.

Coenzyme Q10 facilitates respiratory chain function and hence augments the process of reverse electron transport. This process plays a key role in the thermogenic effect produced by accelerated fatty acid oxidation.

Biotin induces the upregulation of the enzyme glucokinase (GK) in the pancreas and enhances the process of glucose-induced insulin secretion. In addition, biotin counteracts the effect HCA has on the pancreas. HCA tends to inhibit glucose-induced insulin secretion, while biotin facilitates it by activating GK. Together, HCA and biotin maximize pancreatic fuel homeostasis.

Pyridoxine supplementation augments physiological levels of pyridoxal phosphate. This compound interacts with glucocorticoid receptors to downregulate their activity and hence to diminish glucocorticoid effects throughout the body. This action tends to decrease visceral fat accumulation as well as promote insulin sensitivity. Improved insulin sensitivity contributes to insulin-induced appetite suppression. In addition, a direct consequence of improved insulin sensitivity is a decrease in the activity of insulin like growth factor (IGF), a potent cancer inductive agent.

Chromium improves insulin sensitivity in the central nervous system and skeletal muscle. The former contributes to insulin-induced appetite suppression at the level of the hypothalamus while the latter improves insulin-induced glucose disposal into skeletal muscle. In the present invention, chromium can be added in the form of a non-toxic salt, such as, e.g., chromium diglycinate, chromium arginate, chromium picolinate, and the like.

CLA, alpha lipoic acid, gymnema sylvestre, coenzyme Q10 and magnesium each have insulin sensitizing effects. In this invention, magnesium can be added in the form of a non-toxic salt.

With respect to the amounts of the individual components of the composition of this invention, the term “effective amount” means that amount of the component which, when used in combination with the other components in the composition, will provide the composition with the capability of modulating nutrient partitioning so as to increase oxidation of fat and/or promote increased storage of glycogen.

Preferably, the composition contains an HCA:carnitine weight ratio of from about 1:10 to about 100:1, an HCA:biotin weight ratio of from about 50:1 to about 2500:1, and an HCA:gluconeogenic substrate weight ratio of from about 5:1 to about 1:60.

2. Additional Ingredients

The basic mechanism of nutrient partitioning, lipid reduction, and weight loss described in the above-cited patent involves the activation of hepatic thermogenesis. This CIP application extends these concepts and includes additional agents that function to augment the metabolic actions described in the prior patent. These involve two basic processes. The first is the accelerated production of high energy electrons in liver mitochondria. The second coupled process involves the transport of these high energy electrons from the mitochondrial into the cytosolic compartment where their energetic status is “discounted” by passage through futile energy consuming cycles. This forms the basis for the hepatic thermogenesis.

Enhancement of these same processes occurs via inclusion of additional substances as described below. They may be addressed in three conceptual categories. Category I involves enhanced fatty acid oxidation. Category II involves upregulation of thermogenic pathways. Category III involves downregulation of neuronal appetite centers.

Category I

A) Medium Chain Triglycerides (MCT) with fatty acid backbones containing 6 to 14 carbon atoms or their individual fatty acid analogues or metabolic precursors

B) Sesame seeds or their derivates; Sesamin and/or its epimer Episesamin

C) Caffeine

D) Forskolin

Typical MCTs are octanoic acid, decanoic acid, and hexanoic acid. Generally, commercially available MCT includes a mixture of several or all of the MCTs. MCTs are oxidized in mitochondria. Their passage from the cytosol into the mitochondrial compartment does not depend upon activity of carnitine palmitoyl transferase (CPT) activity. Hence, when CPT activity is low, MCTs are still actively oxidized in liver. This provides a continuous source of high energy electrons which may then be transported into the cytosol to enter the futile cycles. This enhanced electron flux contributes to hepatic thermogenesis. In addition, MCTs downregulate both acetyl-CoA carboxylase (ACC) and citrate lyase (CL), thus activating CPT indirectly by reduction of cytosolic malonyl-CoA concentration. This serves to further enhance long chain fatty acid oxidation.

Sesamin and episesamin have been shown in rodent liver to downregulate lipogenic (fat-synthesizing) enzymes, to increase mitochondrial number, to enhance CPT enzyme expression and to increase fatty acid oxidation. These all enhance Category I actions and result in the increased generation of mitochondrial high energy electrons.

In liver cells, cyclic adenosine monophosphate (cAMP) augments CPT transcription and downregulates ACC activity via enhanced enzymic phosphorylation. The compound forskolin enhances the hepatic cAMP concentration. Caffeine, a methylxanthine, inhibits the breakdown of cAMP. Hence, they both augment cAMP levels and act synergistically in this regard. This elevation of cAMP increases fatty acid oxidation in liver cells. A unique benefit of their combined administration is the lack of any elevation in blood pressure.

Category II

A) 7-keto dehydroepiandrosterone (7-keto DHEA)

B) Green tea extract containing epigallocatechingallate (EGCG)

C) Capsaicum

In rodents, 7-keto DHEA potently induces the hepatic mitochondrial enzyme glycerol-3-phosphate dehydrogenase. This enzyme is rate limiting for the glycerol-3-phosphate shuttle. This shuttle drives the glycerol-3-phosphate transmembrane thermogenic electron cycle. Green tea leaf extract/EGCG lowers respiratory quotient (RQ) which reflects enhanced fat oxidation. It has also been shown to activate thermogenesis. Capsaicum, the major principle in capsicum fruits (such as hot red peppers), also augments fat oxidation and thermogenesis likely via , adrenergic mechanisms.

Category III

5-hydroxytryptophan (5-HTP)

5-HTP is a serotonin precursor which has been shown to diminish appetite via central actions especially for carbohydrate rich “comfort foods.” This effect acts synergistically in producing desirable nutrient partitioning and energetic control.

The composition according to the invention preferably also includes one or more nutritional supplements, such as vitamins and minerals. The B vitamins are especially preferred, particularly for difficult excess intracellular lipid storage, such as hepatic steatosis/steatohepatitis

The composition of this invention preferably includes a pharmaceutically acceptable carrier. As used herein, the term “pharmaceutically acceptable carrier” is meant to include one or more pharmaceutically suitable, inactive excipients, carriers, diluents, adjuvants, and lubricants. Non-limiting examples of inactive excipients, carriers, diluents, lubricants, and adjuvants which can be used in the composition of the present invention include: cellulose, substituted cellulose, calcium carbonate, dicalcium phosphate, starches, lactose, modified food starches, dextrose, calcium sulfate, magnesium carbonate, magnesium stearate, stearic acid, glycerin, vegetable oils, polysorbates, lecithin, silicium dioxide, food glaze, talc, croscarmellose sodium, povidone, water and gelatin. Additional inactive excipients, carriers, diluents, lubricants and adjuvants which may be used with the active-ingredient composition of this invention are disclosed in the Handbook of Food Additives (CRC Press), which is incorporated by reference herein in relevant part.

The pharmaceutically acceptable carrier can be present in any conventional amount used in an orally administered composition.

3. Specific Applications

Set forth in Table I below is a preferred embodiment of the basic orally administered composition (excluding inactive ingredients) of this invention. Table II sets forth a more preferred embodiment of the basic composition. The amounts recited in Tables I and II represent the preferred daily dosage of the ingredients listed.

TABLE I
PREFERRED BASIC COMPOSITION

| Orally Administered Composition | Preferred Daily Dosage |
|---|---|
| Gluconeogenic Substrate | 1-75 grams |
| HCA | 0.2-8 grams |
| Carnitine | 10 milligrams-10 grams |
| Biotin | 1-25 milligrams |
| Chromium | 100 micrograms-2 milligrams |
| CLA | 50 milligrams-20 grams |
| EPA (alone or as a component of fish | 10 milligrams-10 grams |
| body oil) | |
| Coenzyme Q10 | 5-500 milligrams |
| Alpha Lipoic Acid | 25-2000 milligrams |
| Magnesium | 200-1600 milligrams |
| Pyridoxine | 25-400 milligrams |
| Gymnemic Acid (from gymnemic | 20-2000 milligrams |
| sylvestre) | |

TABLE II
MORE PREFERRED BASIC COMPOSITION

| Orally Administered Composition | Preferred Daily Dosage |
|---|---|
| Gluconeogenic Substrate | 1-30 grams |
| HCA | 0.5-5 grams |
| Carnitine | 50-5000 milligrams |
| Biotin | 2-10 milligrams |
| Chromium | 400-1000 micrograms |
| CLA | 1-10 grams |
| EPA (alone or as a component of fish body oil) | 50-5000 milligrams |
| Coenzyme Q10 | 10-400 milligrams |
| Alpha Lipoic Acid | 50-800 milligrams |
| Magnesium | 400-1200 milligrams |
| Pyridoxine | 100-300 milligrams |
| Gymnemic Acid (from gymnemic sylvestre) | 75-500 milligrams |

According to the present invention, there is added to the basic composition one or more of the additional ingredients listed in TABLE III. The amounts listed in TABLE III include both a daily dose range and the preferred daily dose.

TABLE III
ADDITIONAL INGREDIENTS

| Agent Dose | Daily Dose Range | Preferred Daily Dose |
|---|---|---|
| CATEGORY I | | |
| MCT(or fatty | 100 milligrams-200 | 5-60 grams |
| acids/derivatives) | grams | |
| Sesamin | 1-1000 milligrams | 10-400 milligrams |
| Episesamin | 1-1000 milligrams | 10-400 milligrams |
| Caffeine | 1-1000 milligrams | 20-500 milligrams |
| Forskolin | 1-1000 milligrams | 20-300 milligrams |
| Sesame seeds (or derivative | 10 milligrams-30 | 100 milligrams-10 |
| products) | grams | grams |
| CATEGORY II | | |
| 7-keto DHEA | 1-1000 milligrams | 20-200 milligrams |
| EGCG (in green tea extract) | 5-900 milligrams | 30-500 milligrams |
| Capsaicum | 0.1-100 milligrams | 0.4-50 milligrams |
| CATEGORY III | | |
| 5-HTP | 1-2000 milligrams | 50-750 milligrams |

One or more of the agents in TABLE III are combined in the appropriate amounts and relationships depending upon the situation by one skilled in the art in combination with agents described in the prior patent application and patent and summarized in Section 1 above, and preferably with one of the combinations listed in TABLE I or TABLE II. Delivery systems are similar to those listed in the prior patent application and patent.

The compositions presented in the tables above are preferably in the form of an orally administered composition, e.g., powder, chewable wafer, tablet, regular or compressed capsule, etc., wherein the amounts listed may be divided into two, or more, portions which in combination constitute a single “serving” or “unit dose” of the composition. Each serving preferably is taken with 8 ounces of water.

The method of this invention involves the steps of administering to a human on a daily basis for a therapeutically effective period of time an effective amount of the composition of this invention. The composition is administered orally or parenterally, preferably orally.

As used herein with respect to the amount of the composition used in the method of this invention, the term “effective amount” means an amount sufficient to modulate nutrient partitioning in the body so as to increase oxidation of fat and/or increase storage of glycogen. Preferably, the active-ingredient composition (not including inactive ingredients) of this invention is administered in a per serving (e.g., daily) dosage of at least about 1 gram, more preferably from about 1 gram to about 200 grams, most preferably from about 4 grams to about 20 grams. When inactive ingredients are present in the composition, the inactive ingredients of the composition can be present in any conventional amount used in orally or parenterally administered compositions.

The term “therapeutically effective period of time” with respect to the administration of the composition in the method of this invention means that period of time sufficient to modulate nutrient partitioning in the human. Preferably, the composition of this invention is administered on a daily basis for a period of at least three weeks, more preferably at least six weeks.

As stated above, oral administration is accomplished by ingesting the composition, preferably with water. The orally administered composition of this invention can be in any conventional form including, e.g., capsules (regular or compressed), tablets, chewable wafers, elixirs, powders, granules, suspensions in water or non-aqueous media, sachets, etc. Powder, tablet, and chewable wafer forms are most preferred.

Alternatively, the composition can be administered parenterally.

As stated previously herein, the method of the invention preferably involves the administration, preferably daily oral administration, of the above-described composition in conjunction with a specific dietary plan in a synergistic format. The dietary plan preferably involves multiple small meals, each reflecting the overall macronutrient composition of the diet. Carbohydrate content is low as is the glycemic index. Protein content is high. In the dietary regimen followed in the present invention, the glycemic index is preferably less than 60, more preferably less than 45; the carbohydrate content will constitute less than 50%, more preferably 0%-50%, most preferably about 7%-40% of the calories consumed on a daily basis; and the protein intake constitutes preferably at least 20%, more preferably about 20%-40%, most preferably about 25-35% of total daily caloric intake. The number of meals is preferably 2, more preferably 4 to 6, per day. Adequate fluid intake is recommended to insure excellent hydration.

As stated above, the composition of this invention is preferably administered on a daily basis. However, if desired, the composition can be administered on a non-daily basis, e.g., every other day. The frequency of administration will depend on how fast the individual wishes to lose weight. The more frequent the composition is administered, the faster the weight loss. Thus, daily administration of the composition will result in faster weight loss than non-daily administration.

Low glycemic index, low carbohydrate, high protein diets have the physiologic effect of inducing low serum insulin (I) levels and low serum insulin/glucagon (I/G) ratios. Both of these parameters act to disinhibit hepatic fatty acid oxidation by decreasing malonyl CoA levels and desensitizing CPT to inhibition by malonyl CoA. On the other hand, if a high carbohydrate diet (greater than 50% carbohydrates) is consumed, average daily insulin levels and the I/G ratio are increased, thereby adversely impacting CPT activity and acting to inhibit fat oxidation.

The method of this invention preferably further includes an exercise program. Preferably, the exercise program will be followed at least 2 days a week and more preferably 3 to 5 days per week. The exercise program should preferably include components of aerobic and resistance training as tolerated by the individual in need thereof.

The exercise program augments fatty acid oxidation during the period of active exercise as well as inducing a fall in RQ post exercise. Insulin sensitivity is increased after each bout of exercise as well as with exercise training. Exercise programs produce changes parallel to those described herein which augment insulin sensitivity, facilitate energy expenditure, and reduce RQ for an extended period of time post exercise.

The invention may further include a stress reduction program designed to diminish glucocorticoid activity. By downgrading glucocorticoid activity, the stress reduction program acts to improve insulin sensitivity and decrease visceral obesity. Utilizing similar mechanisms, pyridoxine augments both these actions. The stress reduction program may involve any activity that lowers glucocorticoid levels. Non-limiting examples of such activities include relaxation, getting a massage, acupuncture, psychotherapy, meditation, taking a sedative, and the like.

The method of the present invention may also include a blood donation program. Blood may be given about every 56 days. This produces a fall in both serum ferritin levels and iron stores within the body which together decrease oxidative stress and improve insulin sensitivity. The amount of blood donated will depend on the individual's serum ferritin levels. Generally, the frequency and amount of blood donated should be such as to provide a serum ferritin level of from about 25 to about 50 nanograms/milliliter of serum.

The method of this invention results in marked increases in fat oxidation and/or glycogen storage while simultaneously minimizing fat synthesis and storage.

Without being bound by any particular overall mechanistic explanation of the invention, the effects of the present invention upon hormone levels and ratios (i.e., low I level, low I/G ratio) facilitate the release of fatty acids for presentation at the hepatocyte mitochondrial membrane. At this locus resides the enzyme CPT. As stated previously herein, CPT is the rate-limiting enzyme in the oxidation of activated long chain fatty acids. Carnitine represents the essential cofactor for CPT and is generally in the subsaturating range in the liver. Exogenous supplementation of carnitine augments CPT activity. Low I levels and a low I/G ratio tend to cause carnitine uptake and concentration in hepatocytes, which in turn acts to increase CPT activity in the liver.

Malonyl CoA, a potent inhibitor of CPT, is a cytosolic metabolite derived from citrate. The cytosolic enzymes CL and ACC are involved in metabolizing cytosolic citrate to acetyl CoA and then on to malonyl CoA, respectively. HCA is a potent competitive inhibitor of CL, thus acting to decrease malonyl CoA levels. EPA/fish oil decreases the activity of both CL and ACC and makes CPT less sensitive to the inhibitory effects of malonyl CoA. The effect of the low I level and low I/G ratio resulting from the dietary plan additionally diminishes the activity of CL and ACC and have a similar desensitizing effect on CPT to the inhibitory action of malonyl CoA. CLA also serves to activate CPT. The action derived from the combined effect of these mechanisms profoundly disinhibits fatty acid oxidation.

Hepatic oxidation of fatty acids to acetyl CoA proceeds independently of the rate of generation of ADP by the liver. This induces the production of mitochondrial acetyl CoA and ATP at high rates. Reducing equivalents in the form of NADH and FADH₂ are also abundantly generated. These effects combine to upregulate reverse electron transport—a highly thermogenic process. In addition, the enzyme PC is activated by the production of high levels of acetyl CoA. At the same time, the enzyme PDH is inhibited by the high ATP levels. Together, these actions channel substrate into the gluconeogenic pathways and away from fat synthetic pathways. The reducing equivalents generated by activation of the fatty acid oxidation process also drive gluconeogenesis. The gluconeogenic process is also thermogenic and is coupled with replenishment of hepatic glycogen stores via the indirect pathway as well as enhanced hepatic glucose output—a process that provides a continuous slow time release source of serum glucose. The gluconeogenesis substrate used in the invention acts as substrate for the gluconeogenic pathway, further acting to facilitate gluconeogenesis. Because the gluconeogenesis substrate is provided exogenously, it tends to spare muscle protein breakdown, thus acting in an anticatabolic fashion and promoting expansion of lean tissue mass. The net effect of this combination of metabolic actions is to profoundly suppress appetite while increasing energy levels, and at the same time oxidizing fat at high rates in a thermogenic fashion. These combine to decrease energy intake while enhancing energy expenditure. This causes a significant depression of RQ as a reflection of the profound alteration in fuel homeostasis and nutrient partitioning which are induced. This lowers the risk of recurrent weight gain following prior weight loss.

### EXAMPLE

To illustrate the invention, an exemplary embodiment in accordance with the invention that provides excellent weight loss without side effects was formulated. This particular formulation also is particularly effective in treating hepatic steatosis/steatohepatitis. Daily administration of a composition containing the following ingredients is recommended for a period of time necessary to achieve a desired result, typically for a time period in a range of from two months to twelve months, or until resolution of the obesity condition, hepatic fat accumulation, inflammation or other dysfunction. Two to three administrations per day are preferred. One composite daily dose contains:

| | | |
|---|---|---|
| Coenzyme Q10 | 100 | mg |
| R alpha lipoic acid | 300 | mg |
| Eicosapentanoic acid (EPA) | 1 | g |
| Trimethylglycine | 500 | mg |
| Phosphatidyl choline | 3 | g |
| S-adenosyl methionine (SAMe) | 200 | mg |
| Carnitine | 500 | mg |
| Aspartic acid (Aspartate) | 2 | g |
| Vitamin B1 | 25 | mg |
| Vitamin B2 | 25 | mg |
| Vitamin B3 | 25 | mg |
| Vitamin B5 | 25 | mg |
| Vitamin B6 | 25 | mg |
| Folic acid | 800 | mcg |
| Biotin | 1 | mg |
| Hydroxycitric acid | 500 | mg |
| Vitamin B12 | 1 | mg |
| MCT | 10 | g |
| EGCG (in green tea extract) | 250 | mg |
| 5-HTP | 500 | mg |

The clinical condition hepatic steatosis/steatohepatitis, as well as the primary and secondary chemical actions and physiologic functionalities of each of the available agents, were considered in selecting agent composition, amounts and ratios. EPA is a beneficial agent for therapy of hepatic steatosis/steatohepatitis, but also facilitates fat burning by other actions that include the inhibition of acetyl CoA carboxylase (ACC) and the activation of carnitine palmitoyl transferase (CPT). Increased fat burning, especially in the liver, lowers the intracellular hepatocyte fat content and ameliorates the condition. Coenzyme Q10, in addition to modulating electron flux, acts as a potent antioxidant with anti-inflammatory actions. It also improves activity of the electron transport chain, thus upregulating reverse electron transport. This discounts the energy status of electrons, further speeding up fat burning as well as oxidizing the Q-couple. This decreases the generation of oxidative stress, thereby indirectly downregulating the hepatic inflammatory process. Aspartate, another redox-active agent, additionally increases hepatic fat burning by upregulating futile, thermogenic carbohydrate cycles, which decreases the intra-cellular fat burden. This further downregulates the inflammation typically present under these conditions. Carnitine also is used as a cofactor for the transport of activated long-chain fatty acids into the mitochondria where they undergo complete oxidation. B vitamins, in addition to their primary actions as described above, are useful cofactors for many of the enzymatic pathways involved in the overall therapeutic process. As discussed above, MCT is a potent fat oxidation agent, especially in the liver. Green tea extract activates thermogenisis. 5-HTP diminishes appetite, particularly for rich foods that are often problematic for individuals with hepatic steatosis/steatohepatitis. These numerous individual modulations act synergistically to maximize hepatic fat burning, further downregulating hepatic fat stores and associated inflammation.

The selection of the agents of the Example exemplifies how methodical selection of agents facilitates the synergy deriving from the combined activity of the primary (related to fatty acid oxidation and handling of high-energy electrons) and secondary (synergistic actions unrelated to the primary effects) actions of the agents. By combining the agents into a formulation that merges the effects with the other additional, implicit, synergistic, beneficial functions of the specifically chosen agents, the functionality of the formulation is maximized.

Prior art formulas sometimes included individual isolated agents known in the prior art to improve specific global clinical endpoints, such as hepatic fat content. Nevertheless, because the prior art failed to consider the detailed molecular mechanisms involved, including the primary and secondary interactions at the cellular level, the prior art did not anticipate the additional beneficial secondary actions included herein in accordance with the invention. Such benefits result from novel considerations of metabolic molecular actions which are not otherwise apparent. The prior art did not recognize or teach the molecular synergies described in this specification. These include, but are not limited to, numerous influences of the redox state on metabolic pathways, and the detailed mechanisms available for the therapeutic modulation of the redox state at many levels and many sites. The selection and application of the interactions, along with the ability to intercede in metabolic pathways and mechanisms in controlled and precise ways, is a benefit of embodiments in accordance with the invention.

3. Use In Foods, Beverages, Condiments, Spices and Salad Dressings

As described in detail in U.S. patent application Ser. No. 10/890,067, which is hereby incorporated by reference to the same extent as fully disclosed herein, the above-described compositions can also be administered by incorporating them into a food, beverage, condiment, spice or salad dressing.

In the preferred embodiment of the invention, the supplement portion of the food, beverage, condiment, spice or salad dressing/supplement combination, is not just any fortification thought to be essential or useful in reducing lipid storage, but comprise a combination of ingredients that have been clinically demonstrated, individually or together, to reduce lipid storage.

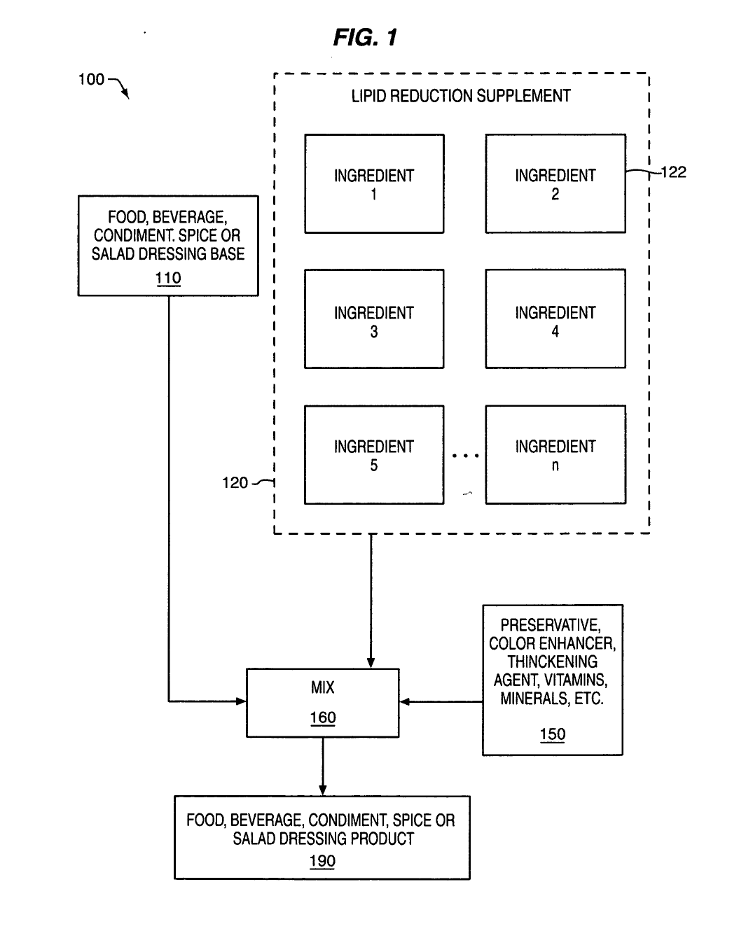

FIG. 1 illustrates the food, beverage, condiment or spice, or salad dressing product 190 according to the invention and the method of making it. The food, beverage, condiment or spice, or salad dressing with lipid reduction supplement product includes a food, beverage, condiment or spice, or salad dressing base 110 and a lipid reduction supplement 120 as discussed above. Optionally, it may also include other non-supplement ingredients 150, such as preservatives, color enhancers, thickening agents, vitamins, minerals, or other such additive ingredients. Food, beverage, condiment or spice, or salad dressing 110, lipid reduction supplement 120, and any other desirable ingredient 150 are mixed at 160 to create food, beverage, condiment or spice, or salad dressing product 190.

In the following, we will disclose a number of different food, beverage, condiment or spice, and salad dressing bases that have been found to be useful to make the food, beverage, condiment, spice or salad dressing product 190. However, it should be understood that these are exemplary, that is, only illustrative, and not intended to be exhaustive.

In this disclosure, all terms that relate to the food, beverage, condiment or spice, or salad dressing component have the meaning commonly used in the food. beverage, condiment, spice and salad dressing arts, respectively. “Food” is defined herein as “material consisting essentially of protein, carbohydrate, and fat used in the body of an organism to sustain growth, repair, and vital processes and to furnish energy. “Food” includes appetizers. Preferably, the word “food” herein is limited to foods as defined above in solid form, to distinguish foods from beverages, though it also includes some liquids that are commonly thought of as foods and not as beverages, such as vegetable oils, syrups, and soups. In this disclosure, “condiment” means something used to enhance the flavor of food; especially, a pungent seasoning. Spices include any of various aromatic vegetable products (as pepper, cinnamon, nutmeg, mace, allspice, ginger, cloves) used in cookery to season food and to flavor foods (as sauces, pickles, cakes) or combinations thereof. Condiments do not include salad dressing, since salad dressings are considered to be a separate category herein. In this disclosure “beverage” means a liquid for drinking, that is, a liquid that is typically drunk for the purpose of refreshment, and specifically does not include water, medicines, or liquid supplementary substances.

Similarly, all terms that relate to the supplement component have the meaning they commonly have in the supplement art. In general, referral to a specific ingredient of a supplement is to be understood as referring to all forms of that ingredient. For example, the term “alpha lipoic acid” (ALA) or “lipoic acid” refers to ALA, lipoic acid, thioctic acid, R alpha lipoic acid, and racemic mixtures thereof. As another example, a salt is interchangeable with its corresponding acid, e.g. aspartate and aspartic acid are interchangeable. Similarly, when other ingredients may exist in various forms, a listing of one form refers to all the various forms, unless expressly noted otherwise.

The terms “food base ”, “beverage base”, “condiment or spice base”, or “salad dressing base” as used herein are intended to include any and all low-fat, low-carbohydrate, or “lite” foods, beverages, condiments, spices and salad dressings also. For example, a low-fat or light salad dressing may be derived from any of the salad dressing bases given below by substituting water and a thickening agent, such as xanthan gum, for all or a portion of the oil given in the formulation, or by other substitutions and formulations known in the salad dressing art. The invention includes the combination of such low-fat foods with the specialized supplements according to the invention.

All of the supplements discussed herein are supplements that are known in the metabolic and medical literature, and thus the dosages that are useful are known in these arts. It is not possible herein to reproduce the vast quantity of literature that addresses dosages of particular supplements that have been found to be useful. However, we have provided exemplary dosages to provide illustrative examples. In all cases, the supplement is present in an amount in excess of the amount of the supplement present naturally in the particular food, beverage, condiment, spice, or salad dressing. In general, the amount of a supplement to be added to a serving of food, beverage, condiment or salad dressing is the appropriate amount for a daily dosage. In other cases in which it is expected that a food, beverage, condiment, spice or salad dressing, or a combination of these, with the supplement added are expected to be consumed several times a day, the amounts given are essentially halved—that is, it is assumed that the intake of the foods and/or beverages will be twice a day. It should be evident that if particular diets call for ingesting of particular foods, beverages, condiments, spices or salad dressings containing the supplements more than twice a day, then the amounts of ingredients in serving portion should be correspondingly reduced. If a food or beverage amount is such that it is intended for multiple servings, then the amount should also be correspondingly increased. Preferably, the foods, beverages, condiments or spices, and salad dressings are provided with a variety of different dosages that are fractions or multiples of the preferred daily dosage so that a consumer can chose to a dosage that is less or more than the preferred daily dosage as the consumer desires. As an example of how an actual product according to the invention can be varied, exemplary salad dressings are disclosed below. The amounts given for the exemplary salad dressings are the amounts sufficient to fill a small bottle of about twelve to fifteen ounces of weight loss/lipid reduction salad dressing. It is intended that the amounts of the ingredients given in the various salad dressing bases should be mixed with the amounts of the ingredients given in the various lipid reduction supplements. The amounts are designed so that a serving of salad dressing is two ounces. It is assumed that salads are eaten twice a day so that two salads, or four ounces of salad dressing, provide effective daily amounts of lipid reduction ingredients. The various amounts given for salad dressing bases may be varied as known in the art of salad dressings, and the various amounts given for weight loss ingredients may be varied as known in the art of weight loss supplements. Similarly, for the other foods, beverages, condiments, spices and salad dressings. In the following, “tsp” means teaspoon, “tbsp” means tablespoon, and “mg” is milligrams.

3A. Food Bases

The food bases do not include chewing gum, inert ingredients by themselves, or other conventional materials for delivering health supplements. The common definition of food necessarily includes only materials that provide protein, fat, and/or carbohydrates. That is, as discussed above, the definition of food is intended to be narrow and is not to be broadened to include materials not commonly recognized as food.

The preferred foods are vegetables and vegetable food products such as baked beans, tomato-based products like tomato paste, stewed tomatoes, spaghetti sauce, pizza sauce, vegetable oils, fruit and fruit food products such as jellies, jams and syrups, cereals, trail mix, cookies, pasta, flours including wheat, soy, oat, and potato flour, whey, chocolate, candy, tofu, bagels, baked goods such as bread, cakes, pizza dough and mixes for these, pancakes and waffles and mixes for these, soups, trail mix, nutritional bars, peanut butter, potato chips, corn chips, crackers, meats, and meat food products, such as lunch meats, hot dogs and sausage, milk food products, such as ice cream, yogurt, cheese and butter.

3B. Beverage Bases

The term “beverage” is used in its common meaning which does not include water or medicines. Beverages are limited to those which are drunk for refreshment. We divide beverages into non-alcoholic and alcoholic beverages because these are related to very different industries and are thought about differently by those skilled in the art of supplements.

I. Non-alcoholic beverages: The preferred non-alcoholic beverages are soft drinks, tea, coffee, milk, fruit juices, and sports drinks. However, any non-alcoholic refreshment beverage is contemplated by the invention.

II. Alcoholic beverages: The preferred alcoholic beverages are beer, wine, liquors, liqueurs, flavored alcohols, brandies, cocktails, aperitifs, and cordials. However, any alcoholic refreshment beverage is contemplated by the invention

3C. Condiment and Spices Bases

Condiments include anything commonly referred to as a condiment, including steak sauce, mustard, catsup, soy sauce, chili sauce, chip dips, salsa, pickles, horseradish, curry, chili powder, etc.

Spices include anything commonly referred to as a spice including salt, pepper, vinegar, cinnamon, garlic and garlic powder or garlic oil, onion and onion powder or onion oil, etc.

3D. Salad Dressing Bases

Any salad dressing can be used as a base for the invention. We shall use salad dressing to provide detailed examples of the process and product of the invention. In these examples, when vegetable oil is mentioned, any vegetable oil may be used, preferably soybean oil or canola oil.

TABLE A
Oil And Vinegar Salad Dressing Base

½ cup water
½ cup vegetable oil
¼ cup vinegar (red wine vinegar may be used)
1 tsp sugar (optional)
½ tsp salt
½ tsp pepper

Small amounts of spices such as onion powder and/or garlic powder may be added.

TABLE B
Caesar Salad Dressing Base

1 egg, raw
3 tbsp lemon juice
garlic (about ½ teaspoon garlic powder or a large clove)
1 cup olive oil (other vegetable oil may be used)
¼ to ½ cup grated Parmesan or Romano cheese
1 tsp high fructose corn syrup
4 tbls anchovy paste
¼ tsp salt and pepper to taste

TABLE C
French Salad Dressing Base

½ cup vegetable oil
⅓ cup high fructose corn syrup
¼ cup water
3 tbls vinegar
½ tsp salt
½ tsp whey
⅓ tsp modified food starch
pinch paprika

TABLE D
Ranch Salad Dressing Base

½ cup water
½ cup vegetable oil
3 tbls vinegar
3 tbls sugar
1 egg yolk
2 tbls buttermilk
½ tsp salt
½ tsp whey
½ tsp modified food starch
¼ teaspoon malted dextrin

TABLE E
Bleu Cheese Salad Dressing Base

½ cup vegetable oil
¼ cup vinegar
¼ cup water
3 tbls blue cheese
2 tbls high fructose corn syrup
1 egg yolk
1tsp lactic acid

Other salad dressing bases include Russian dressing, Thousand Island dressing, Italian dressing, mayonnaise, balsamic vinaigrette, green goddess, and any other dressing used on salads.

From the above disclosure, and the supplement examples given above, those skilled in the art of foods will understand the invention and how to implement it in a wide variety of foods, beverages, condiments, spices, and salad dressings.

There has been described a novel composition and method for reduction of intra and/or intercellular lipid storage, particularly fat storage. It should be understood that the specific formulations and methods described herein are exemplary and should not be construed to limit the invention, which will be described in the claims below. Further, it is evident that those skilled in the art may now make numerous uses and modifications of the specific embodiments described without departing from the inventive concepts. Consequently, the invention is to be construed as embracing each and every novel feature and novel combination of features present in and/or possessed by the compositions and methods described and by their equivalents.

1. An orally or parenterally administered composition for reducing the storage of lipids in a human, said composition comprising:
an effective amount of the hydroxycitric acid;
an effective amount of carnitine;
an effective amount of biotin;
an effective amount of one or more gluconeogenic substrates selected from the group consisting of: aspartate, lactate, glycerol, and a gluconeogenic amino acid or alphaketo analogue thereof;
an effective amount of eicosapentanoic acid; and
an effective amount of one or more ingredients selected from the group consisting of: medium chain triglycerides (MCT) with fatty acid backbones containing 6 to 14 carbon atoms or their individual fatty acid analogues or metabolic precursors, sesame seeds or their derivative products, sesamin and/or its epimer episesamin, caffeine, forskolin, 7-keto dehydroepiandrosterone (7-keto DHEA), green tea extract containing epigallocatechingallate (EGCG), capsaicum, and 5-hydroxytryptophan (5-HTP).

2. A composition according to claim 1 wherein the composition comprises: a weight ratio of the hydroxycitric acid to the carnitine of from about 1:10 to about 100:1; a weight ratio of the hydroxycitric acid to the gluconeogenic substrate of from about 5:1 to about 1:60; and a weight ratio of the hydroxycitric acid to the biotin of from about 50:1 to about 2500:1.

3. A composition according to claim 1 wherein the gluconeogenic amino acid is selected from the group consisting of: alanine, arginine, asparagine, cystine, glutamine, glycine, histidine, hydroxyproline, methionine, proline, serine, threonine, and valine.

4. A composition according to claim 1 wherein the composition further comprises an effective amount of at least one nutritional supplement.

5. A composition according to claim 4 wherein said nutritional supplement is selected from the group consisting of: chromium, conjugated linoleic acid, coenzyme Q10, pyridoxine, magnesium, and gymnema sylvestre.

6. A composition according to claim 5 comprising: from about 0.2 grams to about 8 grams of hydroxycitric acid, from about 10 milligrams to about 10 grams of carnitine, from about 1 gram to about 75 grams of the gluconeogenic substrate; from about 1 milligram to about 25 milligrams of biotin, from about 100 micrograms to about 2 milligrams of chromium, from about 5 milligrams to about 500 milligrams of coenzyme Q10, from about 50 milligrams to about 20 grams of conjugated linoleic acid, from about 10 milligrams to about 10 grams of eicosapentaenoic acid, from about 25 milligrams to about 400 milligrams of pyridoxine, from about 25 milligrams to about 2000 milligrams of alpha lipoic acid, from about 200 milligrams to about 1600 milligrams of magnesium, and from about 20 milligrams to about 2000 milligrams of gymnemic acid.

7. A composition according to claim 5 comprising: from about 0.5 grams to about 5 grams of hydroxycitric acid, from about 50 milligrams to about 5 grams of carnitine, from about 1 gram to about 30 grams of the gluconeogenic substrate; from about 2 milligrams to about 10 milligrams of biotin, from about 400 micrograms to about 1000 micrograms of chromium, from about 20 milligrams to about 300 milligrams of coenzyme Q10, from about 1 gram to about 10 grams of conjugated linoleic acid, from about 50 milligrams to about 5000 milligrams of eicosapentaenoic acid, from about 100 milligrams to about 300 milligrams of pyridoxine, from about 50 milligrams to about 1200 milligrams of alpha lipoic acid, from about 400 milligrams to about 1200 milligrams of magnesium, and from about 75 milligrams to about 500 milligrams of gymnemic acid.

8. A composition according to claim 1, further comprising a pharmaceutically acceptable carrier.

9. A method for reducing the storage of lipids in a human, comprising orally or parenterally administering to the human, for a therapeutically effective period, a composition comprising:
an effective amount of the hydroxycitric acid;
an effective amount of carnitine;
an effective amount of biotin;
an effective amount of one or more gluconeogenic substrates selected from the group consisting of: aspartate, lactate, glycerol, and a gluconeogenic amino acid or alphaketo analogue thereof;
an effective amount of eicosapentanoic acid; and
an effective amount of one or more ingredients selected from the group consisting of: medium chain triglycerides (MCT) with fatty acid backbones containing 6 to 14 carbon atoms or their individual fatty acid analogues or metabolic precursors, sesame seeds or their derivative products, sesamin and/or its epimer episesamin, caffeine, forskolin, 7-keto dehydroepiandrosterone (7-keto DHEA), green tea extract containing epigallocatechingallate (EGCG), capsaicum, and 5-hydroxytryptophan (5-HTP).

10. A method according to claim 9 wherein the composition is administered on a daily basis to said human.

11. A method according to claim 10 wherein said therapeutically effective period of time is at least three weeks.

12. A method according to claim 10 wherein the effective amount of the composition is at least about 1 gram per serving.

13. A method according to claim 10 wherein the effective amount of the composition is from about 4 grams to about 20 grams per serving.

14. A method according to claim 9 wherein the composition comprises: a weight ratio of the hydroxycitric acid to the carnitine of from about 1:10 to about 100:1; a weight ratio of the hydroxycitric acid to the gluconeogenic substrate of from about 5:1 to about 1:60; and a weight ratio of the hydroxycitric acid to the biotin of from about 50:1 to about 2500:1.

15. A method according to claim 9 wherein the gluconeogenic amino acid is selected from the group consisting of: alanine, arginine, asparagine, cystine, glutamine, glycine, histidine, hydroxyproline, methionine, proline, serine, threonine, and valine.

16. A method according to claim 9 wherein the composition further comprises an effective amount of at least one nutritional supplement.

17. A composition according to claim 16 wherein the nutritional supplement is selected from the group consisting of: chromium, conjugated linoleic acid, coenzyme Q10, pyridoxine, magnesium, and gymnema sylvestre.

18. A method according to claim 17 wherein the composition comprises: from about 0.2 grams to about 8 grams of hydroxycitric acid, from about 10 milligrams to about 10 grams of carnitine, from about 1 gram to about 75 grams of the gluconeogenic substrate, from about 1 milligram to about 25 milligrams of biotin, from about 100 micrograms to about 2 milligrams of chromium, from about 5 milligrams to about 500 milligrams of coenzyme Q10, from about 50 milligrams to about 20 grams of conjugated linoleic acid, from about 10 milligrams to about 10 grams of eicosapentaenoic acid, from about 25 milligrams to about 400 milligrams of pyridoxine, from about 25 milligrams to about 2000 milligrams of alpha lipoic acid, from about 200 milligrams to about 1600 milligrams of magnesium, and from about 20 milligrams to about 2000 milligrams of gymnemic acid.

19. A method according to claim 17 wherein the composition comprises: from about 0.5 grams to about 5 grams of hydroxycitric acid, from about 50 milligrams to about 5 grams of carnitine, from about 1 gram to about 30 grams of the gluconeogenic substrate; from about 2 milligrams to about 10 milligrams of biotin, from about 400 micrograms to about 1000 micrograms of chromium, from about 20 milligrams to about 300 milligrams of coenzyme Q10, from about 1 gram to about 10 grams of conjugated linoleic acid, from about 50 milligrams to about 5000 milligrams of eicosapentaenoic acid, from about 100 milligrams to about 300 milligrams of pyridoxine, from about 50 milligrams to about 1200 milligrams of alpha lipoic acid, from about 400 milligrams to about 1200 milligrams of magnesium, and from about 75 milligrams to about 500 milligrams of gymnemic acid.

20. A method according to claim 9 wherein the composition further comprises a pharmaceutically acceptable carrier.

21. A method according to claim 9, further including having said human follow a dietary regimen involving a glycemic index of less than 60 and daily calorie consumption comprising less than 50% of calories from carbohydrate intake and at least 20% of calories from protein intake.

22. A method according to claim 9 wherein the method further includes having said human follow an exercise program involving aerobic and resistance training.

23. A method according to claim 9 wherein the method further involves having the human donate blood so as to produce a fall in serum ferritin levels and iron stores.

24. A method according to claim 9 wherein the method further involves having the human follow a stress reduction program so a to diminish glucocorticoid activity.

25. A food, beverage, condiment, spice or salad dressing product designed to reduce lipid storage, said product comprising:
a food, beverage, condiment, spice or salad dressing base; and
a supplement comprising:
an effective amount of the hydroxycitric acid;
an effective amount of carnitine;
an effective amount of biotin;
an effective amount of one or more gluconeogenic substrates selected from the group consisting of: aspartate, lactate, glycerol, and a gluconeogenic amino acid or alphaketo analogue thereof;
an effective amount of eicosapentanoic acid; and
an effective amount of one or more ingredients selected from the group consisting of: medium chain triglycerides (MCT) with fatty acid backbones containing 6 to 14 carbon atoms or their individual fatty acid analogues or metabolic precursors, sesame seeds or their derivative products, sesamin and/or its epimer episesamin, caffeine, forskolin, 7-keto dehydroepiandrosterone (7-keto DHEA), green tea extract containing epigallocatechingallate (EGCG), capsaicum, 5-hydroxytryptophan (5-HTP);
said supplement present in an amount in excess of the amount of said supplement naturally present in said food, beverage, condiment, spice or salad dressing base.

26. A product as in claim 25 wherein said base is a beverage base and said beverage is an alcoholic beverage.

27. A product as in claim 25 wherein said base is a food base and said food is selected from the group consisting of: vegetables and vegetable food products such as baked beans, tomato-based products like tomato paste, stewed tomatoes, spaghetti sauce, pizza sauce, vegetable oils, fruit and fruit food products such as jellies, jams and syrups, cereals, trail mix, cookies, pasta, flours including wheat, soy, oat, and potato flour, whey, chocolate, candy, tofu, bagels, baked goods such as bread, cakes, pizza dough and mixes for these, pancakes and waffles and mixes for these, soups, trail mix, nutritional bars, peanut butter, potato chips, corn chips, crackers, meats, and meat food products, such as lunch meats, hot dogs and sausage, milk food products, such as ice cream, yogurt, cheese and butter.

28. A product as in claim 25 wherein said base is a beverage base and said beverage is selected from the group consisting of: soft drinks, tea, coffee, milk, fruit juices, and sports drinks.

29. A product as in claim 25 wherein said base is a condiment or spice base and said condiment or spice is selected from the group consisting of: steak sauce, mustard, catsup, soy sauce, chili sauce, chip dips, salsa, pickles, horseradish, curry, chili powder, salt, pepper, vinegar, cinnamon, garlic and garlic powder, garlic oil, onion and onion powder and onion oil.

30. A composition according to claim 25 wherein the gluconeogenic amino acid is selected from the group consisting of: alanine, arginine, asparagine, cystine, glutamine, glycine, histidine, hydroxyproline, methionine, proline, serine, threonine, and valine.

31. A composition according to claim 25 wherein the composition further comprises an effective amount of at least one nutritional supplement.

32. A composition according to claim 31 wherein said nutritional supplement is selected from the group consisting of: chromium, conjugated linoleic acid, coenzyme Q10, pyridoxine, magnesium, and gymnema sylvestre.
